(19) United States
(12) Patent Application Publication
(10) Pub. No.: US 2012/0295333 A1
(43) Pub. Date: Nov. 22, 2012

(54) SYSTEMS AND METHODS FOR PRODUCING A GAS DISPERSION IN A BIOLOGICAL SUBSTANCE IN A DISPOSABLE VESSEL

(76) Inventor: Alan T. Cheng, Naperville, IL (US)

(21) Appl. No.: 13/475,239

(22) Filed: May 18, 2012

Related U.S. Application Data
(60) Provisional application No. 61/487,804, filed on May 19, 2011.

(51) Int. Cl. C12M 1/04; C12N 1/14; C12N 1/16; C12N 1/20

(52) U.S. Cl. 435/252.1; 435/296.1; 435/254.1; 435/255.1

(57) ABSTRACT

Biological liquid substances in a disposable bioreactor or a disposable fermentor is enriched with a dissolved gas such as oxygen. The gas is provided to the liquid in meeting growth needs of biomass within the disposable vessel. The liquid is processed through a bubble forming element such as a supersonic mixer, a membrane sparger, or the like, to form a gas-liquid dispersion. The dispersion has a high interfacial surface area for facilitating gas dissolution in the liquid. Receptacles of the disposable vessel may be coupled (e.g., magnetically) to one or more processing devices (e.g., motors, pumps, gas sources) outside of the disposable vessel so that components within the disposable vessel are able to perform functions suitable for furthering reaction and/or fermentation processes.

### CROSS REFERENCE TO RELATED APPLICATIONS

The present application claims priority from U.S. provisional patent application Ser. No. 61/487,804 filed May 19, 2011.

### FIELD OF THE INVENTION

Aspects relate to systems and methods for processing a biological or other sensitive liquid substance contained within a vessel, preferably a disposable vessel, to form a dispersion of gas bubbles in the liquid. Such systems and methods may relate to forming a gas-liquid dispersion within a bioreactor or a fermentor.

### BACKGROUND

Oxygen is an essential component for cellular respiration in living organisms that undergo aerobic metabolism. In industrial aerobic fermentation processes, oxygen is commonly provided to organisms, such as bacteria or fungus, grown in a fermentation broth by injecting air from a device submerged in the fermentation broth. A fermentation broth often contains various types of biomass and carbohydrates such as molasses, corn starch, sugar or corn syrup. A number of broth formulations also contain vegetable oil in addition to a range of minerals and nutrients that are necessary or helpful for keeping the biomass healthy and growing.

Dispersions of gases, such as air or oxygen, in liquids can be useful for a number of industrial operations, such as for gas dissolution, gas-liquid reaction and gas stripping applications. A dispersion of small, finely dispersed gas bubbles in a liquid generally exhibits a larger degree of interfacial surface area between the gas and liquid as compared to the interfacial surface area between the liquid and larger gas bubbles. Such an increase in interfacial surface area between the gas and liquid can better facilitate mass transfer of the gas within the bubbles into the liquid, and conversely, transfer of dissolved gas from the liquid into the gas bubbles. As a result, processes that increase interfacial surface area in gas-liquid processes can enhance gas dissolution, gas stripping and other gas reactions.

### SUMMARY OF THE INVENTION

The invention may be characterized as a method of processing a biological liquid substance or other sensitive liquid substance in a vessel comprising steps of: (a) providing a vessel, preferably a disposable vessel, having a first processing region and a second processing region,; (b) providing the biological or other sensitive liquid substance to the first processing region; (c) moving a portion of biological or other sensitive liquid substance from the first processing region to the second processing region; (d) supplying a gas to the portion of biological or other sensitive liquid substance within the second processing region forming a mixture of the gas and liquid; and (e) forming a dispersion of bubbles of the gas within the mixture having characteristics similar to dispersions formed by a supersonic mixer or a membrane sparger.

The disposable vessel may be, for example, a disposable reactor or a disposable fermentor vessel, where the liquid may contain living organisms. A gas is supplied to the liquid for dissolution into the liquid so as to assist proliferation of the living organisms within the disposable vessel. Through use of a bubble forming element, such as a supersonic mixer, a membrane sparger or the like, a dispersion of small bubbles of the gas in the liquid is formed. The dispersion is two-phase in nature where small bubbles of the gas phase are dispersed within a continuous liquid such that the gas may be readily dissolved into the liquid within the disposable vessel. Thus, owing to the high interfacial surface area provided by the small bubbles, a significant amount of gas within the small bubbles of the dispersion may readily dissolve into the liquid.

In an illustrative embodiment, a method of processing a biological liquid in a disposable reactor or fermentor vessel is provided. The method includes providing a disposable vessel containing a biological liquid containing living organisms; supplying a gas to be dissolved into the liquid, at least in part, for use by the living organisms; and forming a dispersion of bubbles of the gas in the liquid to dissolve at least part of the gas in the liquid, the dispersion of bubbles being formed in a way equivalent to that by a supersonic mixer or a membrane sparger.

In a further illustrative embodiment, an apparatus for processing liquid is provided. The apparatus includes a disposable vessel containing a liquid, the liquid containing living organisms; and a bubble forming element selected from the group consisting of a supersonic mixer and a membrane sparger that is arranged to introduce a dispersion of gas bubbles into the liquid in the disposable vessel so as to dissolve at least a portion of the gas in the liquid for use by the living organisms.

Various embodiments of the present invention provide certain advantages. Not all embodiments of the invention share the same advantages and those that do may not share them under all circumstances.

Further features and advantages of the present invention, as well as the structure of various embodiments of the present invention are described in detail below with reference to the accompanying drawings.

### BRIEF DESCRIPTION OF THE DRAWINGS

The accompanying drawings are not intended to be drawn to scale. In the drawings, identical or nearly identical components that are illustrated in various figures are represented by like numerals. For purposes of clarity, not every component may be labeled in every drawing. In the drawings:

FIG. 1 is a schematic representation of a supersonic mixer;

FIG. 2 is a schematic representation of a membrane sparger;

FIG. 3 is a schematic representation of an embodiment of a disposable vessel employing a bubble forming element located outside of the disposable vessel;

FIG. 4 is a schematic representation of another embodiment of a disposable vessel employing a bubble forming element located outside of the disposable vessel;

FIG. 5 is a schematic representation of an embodiment of a disposable vessel employing a bubble forming element located inside the disposable vessel; and

FIG. 6 is a schematic representation of another embodiment of a disposable vessel employing a bubble forming element located inside the disposable vessel.

### DETAILED DESCRIPTION

The inventor has recognized and appreciated that forming dispersions in liquids contained within disposable vessels, such as disposable bioreactors or disposable fermentor vessels, may be advantageous for certain processes for production of biochemical, biopharmaceutical and other biologically derived products or other sensitive products such as synthetic media, small molecule pharmaceuticals and liquid nutrition products.

Liquids disposed within such vessels may include a suitable number of living organisms. To form a dispersion in the liquid contained within the disposable vessel, a gas is first supplied to the liquid to form a mixture of the gas and the liquid. A dispersion of small bubbles of the gas is then formed in the liquid so as to enhance mass transfer between the gas and the liquid. Hence, the gas is easily dissolved into the liquid for gas enrichment in the liquid, and the liquid may be suitably used by the living organisms located within the disposable vessel. Such a dispersion may be formed through use of a bubble forming element in a manner that is equivalent to dispersions formed by a supersonic mixer or a membrane sparger.

In some embodiments, disposable vessels are associated with separate processing regions within which liquid may be suitably processed. For example, a first processing region may be provided as a zone for general bioreaction or fermentation processes within the disposable vessel to take place. Accordingly, the liquid containing the living organisms may be added to the first processing region where mixing and growing of the biomass generally occurs.

However, at times, the liquid in the first processing region can be depleted of nutrients and/or gas necessary for the biomass to grow. To further enrich the liquid with gas, a portion of the liquid within the first processing region of the disposable vessel, the portion which may or may not contain living organisms, is transferred from the first processing region to a second processing region. A gas is supplied to the portion of liquid transferred to the second processing region to form a mixture having a gas phase and a liquid phase. The mixture of the gas and liquid is then processed with a bubble forming element into a dispersion where small bubbles of the gas are dispersed throughout the liquid.

In some embodiments, the mixture of gas and liquid is processed through a bubble forming element such as a supersonic mixer or a membrane sparger to form the dispersion. It can be appreciated that usage of such equipment is not limiting, as a dispersion having characteristics of dispersions formed by processing with a supersonic mixer or a membrane sparger can be formed within the mixture of the gas and liquid using other systems or methods. Once the mixture of the gas and liquid is processed so as to form the dispersion, the dispersion may be further processed and eventually returned back to the first processing region as part of a suitable reaction/fermentation process. As a result, the processed liquid is enriched with a gas, such as oxygen, that better enables the biomass within the disposable vessel to grow.

In some embodiments, the bubble forming element may be located outside of the disposable vessel. In such cases, a side-stream outlet may be provided in the disposable vessel for the liquid to be removed and subsequently processed to add the gas to the liquid forming the gas-liquid dispersion.

However, in other embodiments, the bubble forming element may be located within the disposable vessel. Indeed, the entire second processing region or large portions thereof associated with the disposable vessel may be located within the disposable vessel. As such, the disposable vessel may have receptacles that can be coupled (e.g., magnetically) to one or more processing devices (e.g., motors, pumps, gas sources) outside of the disposable vessel so that components (e.g., agitating elements, gas delivery elements, conduits for permitting liquid flow) within the disposable vessel are able to perform their respective functions. In some cases, it may be advantageous to include the bubble forming element and other processing components within the disposable vessel so as to provide a convenient system for user to initiate a reaction/fermentation process. Rather than having to keep track of processing elements such as the bubble forming element, the user simply needs to couple the disposable vessel to a larger support structure having coupling stations to which the disposable vessel can be attached. Once the reaction/fermentation process is completed, the contents of the disposable vessel may be suitably removed and the disposable vessel, along with its respective components, may be taken away to be disposed of as waste and/or recycled.

Sonic shock waves can be used by a bubble forming element to reduce the size of gas bubbles dispersed in a liquid. For example, subjecting a liquid having large gas bubbles dispersed throughout the liquid to a sonic shock wave can create a significant amount of turbulence that effectively shears the larger gas bubbles into much finer bubbles. In some cases, a gas-liquid dispersion is created from a mixture of liquid and large gas bubbles by combining a gas phase with a liquid phase in close proximity to a venturi shaped flow constrictor, accelerating the gas-liquid combination to supersonic flow velocities, and subsequently decelerating the gas and liquid phases to subsonic velocity. Sequential acceleration and deceleration of the gas-liquid phases over and below the speed of sound produces a sonic shock wave that applies a substantial dispersion-forming force to the gas-liquid mixture.

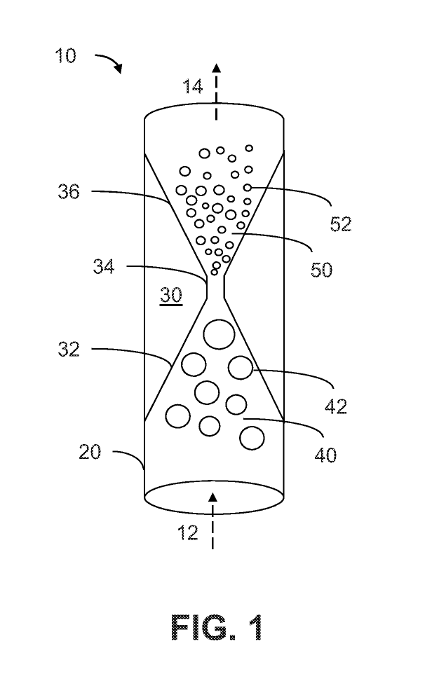

FIG. 1 depicts an illustrative embodiment of a bubble forming element, provided as an in-line supersonic mixer 10, where a two phase gas-liquid mixture 40 is flowed through a conduit 20 to form a gas-liquid dispersion 50 having the same relative compositions of gas and liquid, yet having quite different phase structures. The conduit 20 includes a venturi configuration 30 having a compression region 32, a throat region 34 and an expansion region 36. The venturi configuration 30 may be constructed to have any suitable shapes and dimensions. For example, the compression region 32 and expansion region 36 may have a conical shape and the throat region 34 may be cylindrical in shape, providing for a conical in-line supersonic mixer. Alternatively, the compression and expansion regions 32, 36 may be pyramidal in shape with the throat region 34 having a corresponding shape.

Prior to flow through the supersonic mixer 10, gas is added to the liquid to form a two phase gas-liquid mixture 40 having bubbles 42. The two phase gas-liquid mixture 40 with bubbles 42 enters into conduit 20 through an entrance region 12. As flux of the gas-liquid mixture 40 flows through the compression region 32 of the supersonic mixer 10 and toward the throat 34, the mixture 40 is subject to an acceleration so as to reach supersonic velocities. Though, when the mixture 40 enters through the throat 34 and flows into the expansion region 36, the mixture 40 is decelerated to subsonic velocities. As described above, the abrupt change from supersonic to subsonic flow creates a sonic shock wave, generating substantial forces throughout the mixture 40, shearing the bubbles 42 into much smaller bubbles 52. Once the smaller bubbles 52 are produced in the liquid, a dispersion 50 is formed. The gas-liquid dispersion 50 may exhibit a different consistency than the previous gas-liquid mixture 40 prior to processing through the supersonic mixer. For example, the gas-liquid dispersion 50 may have a more viscous, milky type consistency or a foam-like texture, whereas the gas-liquid mixture 40 prior to being subject to the sonic shock wave would not exhibit such a consistency or texture.

Systems and methods for creating very small bubbles in a liquid by applying a sonic shock wave like those generally described in U.S. Pat. No. 5,211,916 entitled “Stripping System”; U.S. Pat. No. 5,302,325 entitled “In-Line Dispersion of Gas in Liquid”; U.S. Pat. No. 4,867,918 entitled “Gas Dispersion Process and System”; U.S. Pat. No. 4,861,352 entitled “Method of Separating a Gas and/or Particulate Matter from a Liquid”; U.S. Pat. No. 4,931,225 entitled “Method and Apparatus for Dispersing a Gas into a Liquid”; and U.S. Pat. No. 5,061,406 entitled “In-Line Gas/Liquid Dispersion” may be used for producing small bubbles in accordance with aspects of embodiments described herein.

It should be understood that supersonic mixers are not required aspects of the invention, as any suitable bubble forming element may be used in association with the disposable vessel.

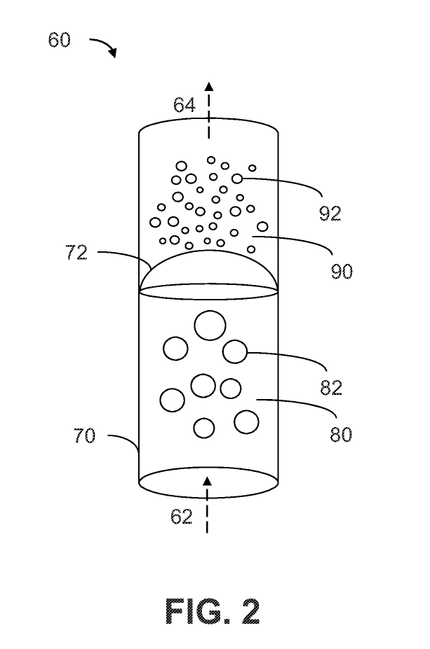

For example, FIG. 2 depicts an illustrative embodiment of a membrane sparger system 60 suitable for use herein as a bubble forming element. A two phase gas-liquid mixture 80 having bubbles 82 is flowed through a conduit 70 having a membrane sparger 72 disposed within the conduit to form a gas-liquid dispersion 90. The gas-liquid dispersion 90 has the same gas-liquid composition as the gas-liquid mixture 80, yet bubbles 92 are substantially smaller than the bubbles 82 having an average size prior to flow of the mixture 80 through the membrane sparger 72. Membrane sparger 72 includes a flexible membrane stretched over a substrate where a suitable number of orifices are located in the membrane through which the gas-liquid mixture 80 flows. In some cases, orifices of the membrane sparger 72 are smaller than the bubbles 82 of mixture 80. Accordingly, as the mixture 80 travels through the membrane sparger 72, the bubbles 82 are sheared into smaller bubbles 92, forming gas-liquid dispersion 90. The orifices of the membrane sparger 72 are spaced sufficiently apart from one another so that bubbles 92 do not coalesce back together forming larger bubbles.

Similarly to that described above regarding dispersions produced from a supersonic mixer, the gas-liquid dispersion 90 may have a different texture or consistency than the previous gas-liquid mixture 80 prior to processing through the membrane sparger 72. For example, the dispersion 90 may be more viscous and/or milky in consistency than the previous mixture 80. The gas-liquid dispersion 90 may exhibit a foam-like texture as well. However, in some cases, the gas-liquid dispersion produced from a supersonic mixer may exhibit a more drastic change in phase structure (e.g., smaller bubbles, higher bubble density) than a dispersion formed from a membrane sparger.

In some embodiments, when a gas-liquid mixture contains living organisms, such as cells, and is formed into a dispersion, it may be preferable to employ a bubble forming element that is gentle on the organisms, while still forming the dispersion. For example, a supersonic mixer may apply an excessive force to cells contained within a gas-liquid mixture resulting in cell death. However, a membrane sparger might apply a comparatively more gentle force to cells disposed within the gas-liquid mixture as compared to a supersonic mixer. In some cases, a membrane sparger may require less pumping pressure throughout the device than the supersonic mixer to function properly. As such, for gas-liquid mixtures that include relatively robust organisms or no organisms at all, larger shear forces such as those that are applied by supersonic mixers may be suitable.

As discussed above, embodiments described herein may be applicable to disposable vessels, such as disposable bioreactors and disposable fermentors. Because of their disposable nature, disposable bioreactors and disposable fermentors are generally smaller than traditional industrial-sized bioreactors and fermentors and, when in use, will have comparably greater cell/organism batch densities.

In some embodiments, a disposable vessel may include a disposable process bag defining a sterile environment and may also include a number of disposable parts within the bag (e.g., probes, sensors, mixing components such as impellers, etc.). A disposable process bag may comprise a flexible bag, made, for example, out of plastic, that is suitably positioned or lined along a support structure. In addition to providing housing for biomaterials and various components suited to carry out different aspects of the process reaction, such as mixing, adding nutrients, sensing of various parameters, injecting air and/or injecting oxygen, the disposable vessel may also include any suitable inlet/outlet port(s) for connecting the vessel to suitable input and/or output sources. For example, upon installing the disposable vessel within a support structure, one or more appropriate gas input sources external to the disposable vessel are coupled to one or more corresponding gas inlet ports within the vessel. Additionally, a sensor included within the disposable vessel (e.g., a dissolved oxygen or dissolved carbon dioxide sensor) may be coupled to an external monitoring device and/or controller once the vessel is installed into a support structure, providing a feedback mechanism for the reaction or fermentation process.

In some embodiments, a disposable vessel is constructed to include a single-use impeller affixed to a lower portion of a flexible plastic bag. The impeller may include an impeller hub mounted on to a post where the impeller hub has an impeller blade suitable for creating currents within a liquid in the bag upon rotation of the impeller. The impeller hub may be coupled to the shaft of a motor that may be provided exterior to or within the support structure of the disposable vessel. In some embodiments, the flexible plastic bag is adapted to be mounted within the support structure such that the motor comes into an aligned arrangement with the impeller hub. Once the motor is suitably aligned with the impeller hub, the motor may drive the impeller hub via a rotating shaft. The flexible plastic bag may also have connection ports for coupling various components within the bag (e.g., sensors, inlets/outlets, etc.) to suitable processing devices external to the bag which enable such components to function properly. In some embodiments, components within a disposable vessel may be coupled to devices exterior to the vessel via a suitable magnetic coupling arrangement.

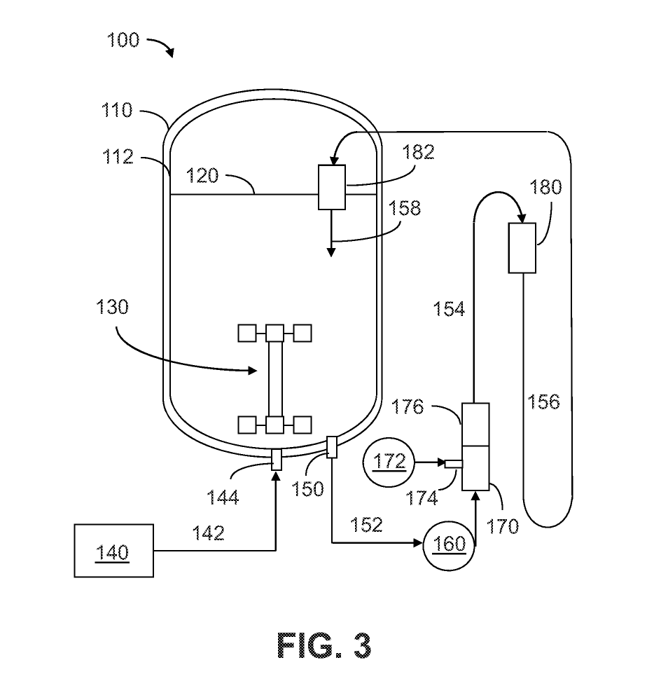

FIG. 3 depicts an embodiment of a system 100 where a disposable vessel 112 containing a liquid 120 is installed in a support tank 110. In some embodiments, the disposable vessel 112 is a disposable bioreactor or a disposable fermentor. As such, the disposable vessel may include a thin plastic bag lining the walls of the support tank 110 where the disposable vessel also includes a number of components useful for running a reaction and/or creating a suitable environment for organisms to grow. Located inside the disposable vessel 112 is an agitation system 130 that includes any appropriate type and number of agitating elements. Gas may be supplied to the liquid 120 within the vessel 112 through an inlet port 144. In this embodiment, a gas source 140 supplies a gas (e.g., air or an oxygen-rich gas) through a conduit 142, such as a pipe, to the inlet port 144.

The region of disposable vessel 112 illustrated in FIG. 3 that includes the zone in which the agitation system 130 is located and where general reaction/fermentation processes occur is considered to be the first processing region. The first processing region generally includes the main chamber of the disposable vessel 112. Though, as discussed above, it may be preferable for a portion of the liquid 120 to be moved from the first processing region to a second processing region where a dispersion of gas bubbles is formed in the removed portion of liquid. The portion of liquid in which a gas-liquid dispersion is formed may or may not be further processed to further enrich the liquid with gas, and may be moved back from the second processing region to the first processing region for consumption by the biomass.

The disposable vessel 112 includes an outlet port 150 through which liquid may flow out of the first processing region of the disposable vessel and into the second processing region. As illustrated, a conduit 152 is in connection with a pump 160 for moving a portion of the liquid 120 to be processed to produce a gas-liquid dispersion. Pump 160 provides an appropriate motive force for liquid to flow through conduits 152, 154, 156, 158 of the second processing region as well as any other processing elements, and then back into the first processing region. It can be appreciated that any suitable arrangement may be provided for moving liquid from 1) the first processing region to the second processing region, 2) through the second processing region, and 3) back from the second processing region into the first processing region. Indeed, a number of pumps may be employed in suitable embodiments for processing liquid for reaction and/or fermentation processes to occur within the disposable vessel. As depicted in FIG. 3, the second processing region may also include a gas delivery element 170, a bubble forming element 176 and redistributing elements 180, 182.

Upon flow of the liquid into the gas delivery element 170, a suitable gas is introduced into the liquid, such as an oxygen-rich gas, or alternatively, air. The gas may originate from a gas source 172 and may be introduced via a suitable inlet port 174. As described herein, an oxygen-rich gas is a gas that has a concentration of oxygen that is greater than the concentration of oxygen found in air. That is, the volume of oxygen in an oxygen-rich gas is greater than about 21%. Oxygen-rich gases may have oxygen having volumes of greater than, for example, 40%, 60%, 80%, or 90% of the gas. In some cases, an oxygen-rich gas may include a gas having a high oxygen purity, such as having oxygen with a concentration of greater than 99% of the gas. When a gas, such as an oxygen-rich gas, is introduced into the liquid, a gas-liquid mixture having appropriately sized bubbles is formed.

The gas-liquid mixture then moves into a conduit containing the bubble forming element 176, for example, a suitable supersonic mixer or membrane sparger. The bubble forming element processes the gas-liquid mixture to form a gas-liquid dispersion. Once formed, the gas-liquid dispersion moves through the remaining portions of the second processing region. As depicted in FIG. 3, the gas-liquid dispersion may move from the bubble forming element 176 through a serpentine piping arrangement provided by conduits 154, 156, 158. Such an arrangement may provide for enhanced gas dissolution (e.g., oxygen dissolution) of the small gas bubbles within the dispersion into the liquid. In some embodiments, the piping arrangement provided by conduits 154, 156, 158 includes a small tube diameter that provides a structure that facilitates dissolution of the gas into the liquid. For instance, the length of travel for the gas-liquid dispersion offered by the piping arrangement may provide for a sufficient amount of time and space for gas to dissolve into the liquid. A generally small tube diameter of the conduits may also assist in maintaining the velocity of the dispersion through the piping arrangement.

As it may be preferable for gas within the dispersion to dissolve into the liquid, it may also be preferable to prevent gas bubbles within the dispersion from substantially coalescing into larger gas bubbles. That is, when gas bubbles coalesce into larger gas bubbles, the interfacial surface area between the gas and liquid is decreased, resulting in a lower dissolution rate of gas into the liquid.

Along the serpentine piping arrangement defined by conduits 154, 156, 158, redistributing elements 180, 182 may be located in close proximity to a bend in the pipe. In some cases, when a liquid having small bubbles dispersed therein travels around a bend in the pipe, the small bubbles may have a tendency to coalesce. Therefore, a redistributing element may be provided so as to minimize coalescing of the small bubbles in the gas-liquid dispersion. In some embodiments, a redistributing element includes a spray device that introduces a high pressure fluid (e.g., gas or liquid) into the dispersion in a manner that imparts kinetic motion to the small bubbles within the dispersion, thus, substantially preventing coalescing. It should be understood that redistributing elements are not required to be sprays, as any suitable redistributing device may be implemented.

As gas within the gas-liquid dispersion dissolves into the liquid, the phase structure of the gas with respect to the liquid may change appropriately. For example, the consistency of the gas-liquid dispersion may change as the dispersion travels through a piping arrangement. In some cases, a substantial amount of gas within the dispersion may dissolve into the liquid, or even may coalesce, such that the two phase gas-liquid composition is no longer a dispersion. For example, a sufficient amount of gas may have dissolved into the liquid such that the dispersion no longer exhibits a viscous, milky consistency. In other cases, although gas within the dispersion may dissolve into the liquid, the two phase gas-liquid composition continues to have a consistency such that it remains a dispersion.

After traveling through to the end of conduit 158, the liquid having gas bubbles dispersed throughout is returned from the second processing region back to the first processing region. In this respect, the liquid that was once removed from the first processing region is enriched with gas (e.g., oxygen) in the second processing region (forming the dispersion) and then re-incorporated into the first processing region to be incorporated in a regular reaction/fermentation process. As such, biomass within the disposable vessel is better enabled to grow as the gas nutrient dissolved in the liquid is more readily available to the biomass than it would otherwise have been had the liquid not been processed to form the dispersion, or even if the gas had simply been injected into the liquid, for example, through a sparger. Since, for some embodiments, the gas-liquid dispersion is further processed (e.g., through conduits and redistributors), it can be appreciated that the gas enriched liquid may or may not have the same dispersion structure as that initially produced from the bubble forming element upon re-introduction back into the first processing region of the disposable vessel.

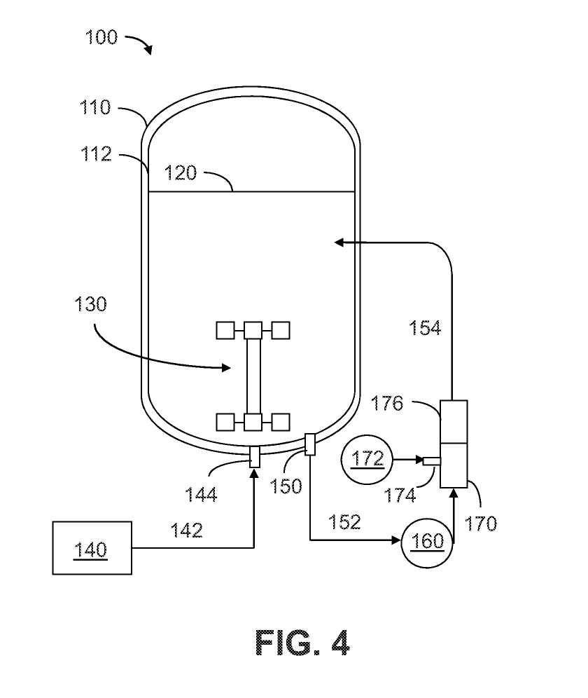

Although provided in FIG. 3, the piping arrangement is not a necessary requirement for the system 100. FIG. 4 depicts an alternative embodiment where liquid 120 is pumped out of the first processing region of the disposable vessel 112 via a gas outlet 150 and into a second processing region having only conduits 152, 154, pump 160, gas delivery element 170 and bubble forming element 176. As the liquid flows into the gas delivery element 170, a suitable gas (e.g., oxygen-rich gas) is introduced into the liquid to form the gas-liquid mixture. The gas-liquid mixture is then transferred to the bubble forming element 176 where a gas-liquid dispersion is formed. The gas-liquid dispersion is then returned back into the first processing region of the disposable vessel 112. For the illustrative embodiment of FIG. 4, although the gas-liquid dispersion does not travel through a piping arrangement that facilitates dissolution of the gas into the liquid, the gas-liquid dispersion still includes a substantial number of small bubbles having a large interfacial surface area that allows for the gas to easily dissolve into liquid.

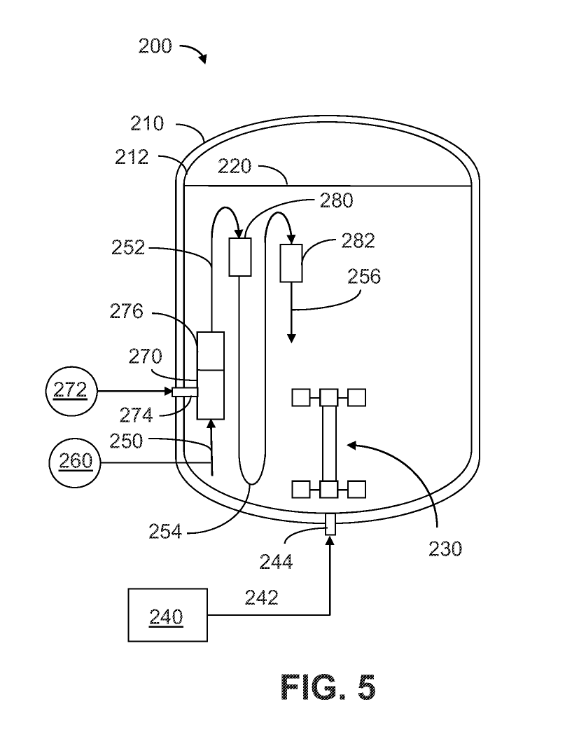

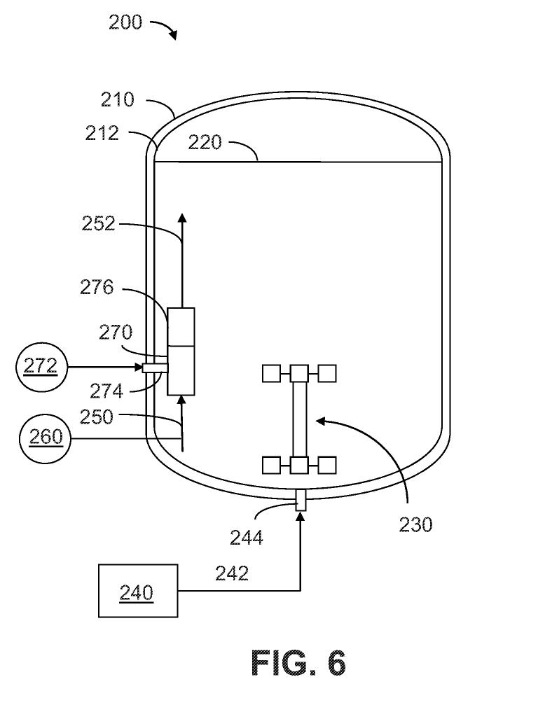

As illustrated above for the embodiments of FIGS. 3 and 4, the first processing region is disposed within the disposable vessel 112 while the second processing region, for the most part, is disposed outside of the disposable vessel 112. That is, the bubble forming element where the liquid is formed into a dispersion having small gas bubbles is located exterior to the disposable vessel 112. Though, it can be appreciated that the bubble forming element where the gas-liquid dispersion is formed may be located interior to the disposable vessel 112, as illustrated in embodiments shown in FIGS. 5 and 6. Despite both first and second processing regions being located within the disposable vessel 112, a portion of the liquid 120 may still be separated from the first processing region to be processed in the second processing region to form the gas-liquid dispersion. After flow through the bubble forming element, the portion of liquid in which a gas-liquid dispersion is formed may or may not be further processed, as desired, and may subsequently be transferred from the second processing region back into the first processing region of the disposable vessel.

In FIG. 5, the system 200 includes a disposable vessel 212 containing a liquid 220. In various embodiments, the disposable vessel 212 is a disposable bioreactor or a disposable fermentor vessel. Accordingly, the disposable vessel may include a thin plastic bag that lines a support tank 210. As described above with respect to FIGS. 3 and 4, the disposable vessel may also include components that may assist in facilitating a reaction and/or creating an environment for organisms to grow (e.g., mixing, inputting gas/nutrients, etc.). The disposable vessel 212 includes an agitation system 230, and a suitable gas (e.g., air or an oxygen-rich gas) is supplied to the liquid 220 via inlet port 244 from a gas source 240 via conduit 242.

Within the disposable vessel 212 is located a first processing region where general steps for reaction/fermentation processes occur (e.g., adding nutrients, mixing, maintaining/adjusting temperature/pH levels, etc.). Within the disposable vessel 212 is also located a second processing region having bubble forming element 276. A portion of the liquid 220 is transferred from the first processing region to the second processing region to form a dispersion of gas bubbles in the transferred portion of liquid. After processing in the second processing region, the liquid is returned back to the first processing region, the transfer and process steps occurring all within the disposable vessel 212.

A conduit 250 is located within the disposable vessel 212 providing a passageway for liquid to move from the main processing chamber of the vessel and to the gas delivery element 270. A pump 260 may be coupled with a region of the disposable vessel (e.g., pump receptacle) so as to provide a suitable motive force for liquid to flow through conduits 250, 252, 254, 256 and other components of the second processing region. While the pump 260 is schematically depicted in FIG. 5 to be located outside of the disposable vessel 212, it can be appreciated that one or more pumps may be located inside and/or outside the disposable vessel 212.

As the liquid enters the gas delivery element 270, a suitable gas such as air or oxygen-rich gas is introduced into the liquid to form a gas-liquid mixture. The gas-liquid mixture is then processed with the bubble forming element 276, such as a suitable supersonic mixer or membrane sparger, located within the disposable vessel 212, to form a gas-liquid dispersion.

The gas-liquid dispersion may then move appropriately through remaining portions of the second processing region, also located inside the disposable vessel 212. As depicted in FIG. 5, the gas-liquid dispersion may travel through a serpentine piping arrangement provided by conduits 252, 254, 256, providing for enhanced gas dissolution (e.g., oxygen dissolution) of the small gas bubbles within the dispersion into the liquid. Suitable redistributing elements 280, 282 may be located near bends in the serpentine piping arrangement inside the disposable vessel 212, provided by conduits 252, 254, 256, so as to minimize coalescing of the small bubbles.

Accordingly, the liquid having initially entered the second processing region through conduit 250 exits the second processing region through conduit 256 and is delivered back into the first processing region, having been enriched with gas (e.g., oxygen). Thus, more of the added gas is dissolved and made available to the biomass within the main chamber of the disposable vessel so as to facilitate growth and proliferation.

FIG. 6 illustrates an embodiment where the disposable vessel 212 does not include a serpentine piping arrangement such as that shown in the embodiment of FIG. 5 in the second processing region. Thus, the second processing region generally includes only conduits 250, 252, gas delivery element 270 and bubble forming element 276. A suitable gas (e.g., oxygen-rich gas) is introduced into the liquid via the gas delivery element 270 to form a gas-liquid mixture, which is then processed to form a gas-liquid dispersion via the bubble forming element 276. The gas-liquid dispersion is then returned back into the first processing region of the disposable vessel 212 for incorporation in subsequent reaction and/or fermentation processes within the main chamber of the vessel.

Where certain components are located inside the disposable vessel, such as parts of a second processing region (e.g., the bubble forming element), components of the disposable vessel may be coupled with one or more devices exterior to the disposable vessel that enable functions within the vessel to occur. For example, as discussed above, an impeller may be located within the disposable vessel and the hub of the impeller may be coupled to a motor located exterior to the disposable vessel having a configuration and power source for driving the impeller blades. The motor may be provided as part of the support tank to which the disposable vessel is installed or may be a separate device altogether for providing functionality to one or more agitating elements within the disposable vessel. Any appropriate coupling arrangement may be employed, such as through a suitable male-female connection port, a magnetic coupling between the impeller hub and the motor, or another suitable arrangement.

Similarly, conduits and other processing elements of the disposable vessel may be placed in a coupling arrangement with an exterior pumping source for moving the liquid between and through elements within the first and second processing regions. It follows that the disposable vessel may have a receptacle to which the exterior pumping source may be suitably coupled for the pumping source to be in a position to drive movement of the liquid within the disposable vessel (e.g., through a gas delivery element and bubble forming element within the second processing region). Any appropriate pump may be used as a pumping source for moving the liquid between and through elements of processing regions within the disposable vessel.

For components of the disposable vessel to which air is injected, such as the gas delivery element of the second processing region or the gas inlet port of the first processing region, the disposable vessel may include a gas input receptacle to which one or more gas supply sources may be coupled for appropriately supplying gas. The gas supply source(s) may be located exterior to the disposable vessel as part of the support tank or may be provided as a separate gas supply device also exterior to the disposable vessel.

Indeed, the disposable vessel may have any suitable number of receptacles corresponding to components where the disposable vessel may be coupled to appropriate processing devices for permitting components within the disposable vessel to function, for example, such that desired reaction/fermentation processes may suitably occur. Such components of the disposable vessel may include agitating elements, gas inlet/outlet ports, conduits/elements through which liquid is to be pumped through, sensors, or any other suitable component that can be activated through a processing source external to the disposable vessel.

In an embodiment, the disposable vessel is magnetically attachable to a receiving area of the support tank for coupling the disposable vessel to devices external to the vessel. Once the disposable vessel is suitably attached to the receiving area, the devices which correspond to particular components within the disposable vessel provide suitable functionality to those components. Once the disposable vessel is removed from the receiving area, the components then cease their respective function(s). It may be advantageous to manufacture such a system so that less expensive components (e.g., conduits, impeller parts, sensors, etc.) may be included within the disposable vessel and more expensive devices (e.g., motors, pumps, controllers) can be located outside of the disposable vessel and experience multiple usages with a number of different vessels.

Aspects described herein may be employed in any appropriate oxidation and/or fermentation reaction mixture. For example, in fermentation reactions, the reaction mixture may include a fermentation broth that generally includes water, nutrients or fermentable constituents such as corn syrup, molasses and glucose, and a biological organism such as bacteria, fungus and/or yeast. Fermentation mixtures may contain additives such as antifoam agents, nitrates or chemicals for pH adjustment, and the like. Some examples of fermentation products that may be produced by methods described herein include antibiotics such as penicillin, erythromycin and tetracycline; organic chemicals such as ethanol, sorbitol and citronellol; organic acids such as citric acid, tartaric acid and lactic acid; amino acids such as L-lysine and monosodium glutamate; polysaccharides such as baker's yeast and xanthan gum; vitamins such as ascorbic acid and riboflavin; and other products including enzymes, insecticides, alkaloids, hormones, pigments, steroids, vaccines, interferon and insulin. Liquid phase oxidation reactions may also be carried out using methods described. For example, such reactions may include the oxidation of toluene to benzoic acid, the oxidation of p-xylene to p-toluic acid, the production of hydrogen peroxide through the oxidation of hydroquinone, the oxidation of toluene to phenol, and the oxidation of paraxylene to terephthalic acid.

Embodiments described herein also provide for more efficient delivery of oxygen to living organisms within certain process vessels, such as disposable fermentors.

In general, industrial sized fermentors may be between 40 and 200 times larger than disposable sized fermentors. For example, certain industrial fermentors have volume of about 200,000 L or greater whereas disposable fermentors may have volume ranging between about 1,000 and 5,000 L. As the volumes of disposable fermentors are typically much smaller than traditional fermentors, the biomass within disposable fermentors, comparatively, can be denser and more than 50% greater, placing limitations on nutrients and oxygen supply. Though, it should be appreciated that systems and methods described herein are applicable to large scale vessels, such as industrial sized fermentors, providing advantageous results.

When systems and methods described herein are implemented for appropriate disposable bioreactors or disposable fermentors, which have high cell/organism batch densities, oxygen consumption and/or power requirements may be reduced. In some embodiments, for high density batches of cells/organisms typically found in disposable fermentors, high levels are recorded for oxygen transfer efficiency, effectively reducing either or both oxygen consumption and/or power requirements.

For purposes described herein, oxygen transfer efficiency (OTE) is measured as the amount of oxygen consumed in a vessel (e.g., reactor or fermentor) compared to the oxygen input into the vessel. For example, a 25% measure of air OTE would indicate that a quarter of the oxygen available through input of air into the vessel had been consumed by the process (e.g., reaction or fermentation) occurring within the vessel. For instance, oxygen consumption for disposable fermentors may be reduced by 40% to 60% or more when embodiments described herein are employed for dispersive oxygen dissolution into the fermentation broth of a disposable vessel. Accordingly, such systems and methods are suitable for batches of biomass having extremely high oxygen demands

Various gas parameters were measured for a disposable fermentor using a system and method of side-stream processing with a supersonic mixer as compared with a disposable fermentor that does not utilize side-stream processing.

In a Conventional Example, a fermentation broth was mixed in a disposable fermentor. Air was injected at an inner mixing region from the bottom of the fermentor directly toward the agitation system, and oxygen-rich gas was injected outside of the inner mixing region via a full-ring sparger.

In the Example, the disposable fermentor incorporates a supersonic mixer similar to the embodiment depicted in FIG. 3. Accordingly, liquid was pumped out of the main chamber of the disposable vessel, an oxygen-rich gas was injected into the liquid and the gas-liquid mixture was formed into a dispersion using a supersonic mixer. The resulting dispersion was then run through a serpentine piping arrangement to facilitate dissolution of the oxygen gas into the liquid. The piping arrangement also had redistributing elements disposed adjacent to bends in the piping to prevent coalescing of the bubbles into larger bubbles. The liquid enriched with oxygen was then returned back into the main chamber of the disposable vessel. A number of measured results for this example as compared with a conventional example are provided in Table 1 below.

TABLE 1

| | Conventional | |
|---|---|---|
| | Example | Example |
| Working Volume (L) | 200 | 200 |
| Air sparge (vvm) | 0.55 | 0.55 |
| O₂ sparge (vvm) | 0.25 | 0.098 |
| O₂ transfer rate (mmol O₂/L/h) | 254 | 254 |
| OTE of air (%) | 21.3% | 21.3% |
| OTE of O₂-rich gas (%) | 28.1% | 71.2% |
| Overall OTE | 25.9% | 44.4% |

Processing a side-stream of liquid from the fermentation broth to incorporate a higher level of dissolved oxygen generally results in a higher oxygen utilization efficiency than would have been obtained if the liquid had not been processed in such a manner. The overall OTE for the Example was greater compared with the Conventional Example, as particularly indicated for the OTE of oxygen-rich gas having been processed with the liquid using the supersonic mixer. Additionally, though not explicitly measured, the power consumption requirements for embodiments incorporating systems and methods of gas enrichment described herein may be less than power consumption requirements for conventional systems and methods without gas enrichment.

Aspects described may be employed to enhance concentrations of dissolved oxygen for fermentation systems, as well as in organic oxidation processes, such as those occurring in bioreactor systems. Suitable methods for gas injection include but are not limited to supersonic gas injection nozzles, simple pipes or gas spargers, orifices, venturi type nozzles or gas-liquid nozzles, depending on the requirements of a given application. If a high oxygen content liquid, or liquid plus oxygen gas is used, the injection device for oxygen addition may be simple pipes, spargers, venturi nozzles or gas-liquid nozzles as desired for the particular application.

Agitation systems described may incorporate any appropriate configuration of agitating elements. Agitating elements included in an agitation system need not be the same. For example, in an embodiment not explicitly illustrated, a Ruston turbine may be employed as a first agitating element and a pitched blade turbine may be used as a second agitating element, or vice versa. Any number of agitating elements may be used in an agitation system, for example, one, two, three, four, or even more agitating elements. Further, agitating elements may share the same shaft and, in some cases, are operated in concert. Though, it can be appreciated that suitable agitating elements can be operated independently, and indeed, may incorporate separate shafts, or might not incorporate a shaft at all. A suitable agitating element may include a paddle that moves back and forth without rotation, or alternatively, a transducer that emits sonic energy into a mixing region. In some embodiments, an agitating element may have a surface that is constructed in a manner such that gas injected toward the agitating element collects behind the surface and is subsequently ejected from the center of the agitating element outward.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the scope of the invention. Accordingly, the foregoing description and drawings are by way of example only.

1. A method of processing a biological liquid substance or other sensitive liquid substance in a bioreactor or fermentor vessel, the method comprising:
providing a bioreactor or fermentor vessel associated with a first processing region and a second processing region, the first processing region located within the bioreactor or fermentor vessel;
providing the biological or other sensitive liquid substance to the first processing region;
moving a portion of biological or other sensitive liquid substance from the first processing region to the second processing region;
supplying a gas to the portion of biological or other sensitive liquid substance within the second processing region forming a mixture of the gas and liquid; and
forming a dispersion of bubbles of the gas within the mixture having characteristics similar to dispersions formed by a supersonic mixer or a membrane sparger.

2. The method of processing a biological or other sensitive liquid substance of claim 1, wherein the bioreactor or fermentor vessel is a disposable vessel and the disposable vessel comprises a disposable bag lining an inner wall of a support structure.

3. The method of processing a biological or other sensitive liquid substance of claim 1, wherein the second processing region is located within the bioreactor or fermentor vessel.

4. The method of processing a biological or other sensitive liquid substance liquid of claim 1, wherein forming the dispersion further comprises flowing the mixture through a supersonic mixer disposed in the second processing region by accelerating the mixture to a supersonic velocity and subsequently decelerating the mixture to a subsonic velocity creating a sonic shock wave.

5. The method of processing a biological or other sensitive liquid substance of claim 1, wherein forming the dispersion further comprises flowing the mixture through a membrane sparger disposed in the second processing region.

6. The method of processing a biological or other sensitive liquid substance of claim 1, wherein moving the portion of the biological or other sensitive liquid substance from the first processing region to the second processing region of the bioreactor or fermentor vessel comprises pumping the liquid substance from the first processing region to the second processing region by coupling a pumping receptacle of the bioreactor or fermentor vessel with a pumping source exterior to the bioreactor or fermentor vessel.

7. The method of processing a biological or other sensitive liquid substance of claim 1, wherein supplying the gas to the portion of liquid within the second processing region of the bioreactor or fermentor vessel comprises introducing gas to a gas input port of the bioreactor or fermentor vessel by coupling a gas input receptacle of the bioreactor or fermentor vessel with a gas supply source exterior to the bioreactor or fermentor vessel.

8. The method of processing a biological or other sensitive liquid substance of claim 1, further comprising operating an agitating element located within the bioreactor or fermentor vessel to mix the liquid disposed within the first processing region of the bioreactor or fermentor vessel by coupling an agitator receptacle of the bioreactor or fermentor vessel with an agitator power source exterior to the bioreactor or fermentor vessel for powering the agitating element.

9. The method of processing a biological or other sensitive liquid substance of claim 1, further comprising moving the liquid through a serpentine pipeline configuration disposed in the second processing region of the bioreactor or fermentor vessel.

10. The method of processing a biological or other sensitive liquid substance of claim 1, further comprising operating a redistributing spray to prevent coalescing of the dispersion of bubbles of the gas within the mixture.

11. An apparatus for processing a biological liquid, the apparatus comprising:
a bioreactor vessel containing a biological liquid, the biological liquid containing living organisms; and
a bubble forming element selected from the group consisting of a supersonic mixer and a membrane sparger that is arranged to introduce a dispersion of gas bubbles into the liquid so as to dissolve at least a portion of the gas in the liquid for use by the living organisms.

12. The apparatus for processing liquid of claim 11, wherein the bubble forming element is located within the bioreactor vessel.

13. The apparatus for processing liquid of claim 11, wherein the bioreactor vessel is a disposable bioreactor vessel.

14. The apparatus for processing liquid of claim 13, wherein the disposable vessel further comprises a magnetic coupling element for coupling a receptacle of the disposable bioreactor vessel to a source exterior to the disposable bioreactor vessel for processing the liquid within the disposable bioreactor vessel.

15. The apparatus for processing liquid of claim 13, further comprising a support structure for the disposable bioreactor vessel, wherein the disposable bioreactor vessel comprises a disposable bag lining an inner wall of the support structure.
